(19) United States
(12) Patent Application Publication
(10) Pub. No.: US 2014/0357828 A1
(43) Pub. Date: Dec. 4, 2014

(54) MODIFIED BETA-AMINO ACID ESTER (ASPARATE) CURING AGENTS AND THE USE THEREOF IN POLYUREA TISSUE ADHESIVES

(71) Applicant: Medical Adhesive Revolution GmbH, Aachen (DE)

(72) Inventors: Christoph Eggert, Koln (DE); Heike Heckroth, Odenthal (DE)

(73) Assignee: Medical Adhesive Revolution GmbH, Aachen (DE)

(21) Appl. No.: 14/369,744

(22) PCT Filed: Jan. 4, 2013
(86) PCT No.: PCT/EP2013/050082

§ 371 Date: Jun. 30, 2014

(30) Foreign Application Priority Data
Jan. 9, 2012 (EP) 12150419.5

(51) Int. Cl. A61L 24/04; C08G 18/48; C08G 18/73

(52) U.S. Cl. CPC A61L 24/046; C08G 18/73; C08G 18/4887
USPC 528/76; 560/171

(57) ABSTRACT

The present invention relates to a compound of a formula (I)

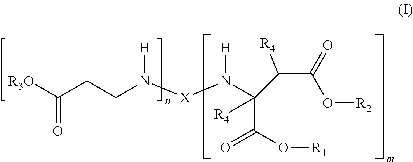

in which
R₁, R₂, R₃ are respectively independent equal or various organic radicals that have no Zerewitinoff active hydrogen,
R₄ are independently hydrogen, equal or different organic radicals, having no Zerewitinoff active hydrogen, or together form an unsaturated or aromatic ring, which can potentially contain heteroatoms, wherein R₄ have no Zerewitinoff active hydrogen,
X is a linear or branched, potentially even a substituted organic radical in the chain with heteroatom, which does not have Zerewitinoff active hydrogen,
n 0<n≦2 and
m 0≦m<2,
   wherein n+m=2. The invention also relates to a process for producing a compound of said formula (I) as well as a polyurea system containing such a compound.

The present invention relates to a beta-amino acid ester, particularly a beta-amino acid ester modified aspartate, a process for the production thereof, as well as the use of this compound as a hardener for the production of polyurethane ureas or polyureas, particularly for adhesives.

Tissue adhesives are commercially available in various forms. This includes the cyanoacrylates, Dermabond® (octyl-2-cyanoacrylate) and Histoacryl Blue® (butyl cyanoacrylate). Cyanoacrylates, however, require dry subsurfaces for efficient adhesion. These types of adhesives fail in the case of severe bleeding.

Biological adhesives, such as BioGlue®, a mixture of glutaraldehyde and bovine serum albumin, various collagens and gelatin-based systems (FloSeal®) as well as fibrin adhesive (Tissucol), are available as an alternative to cyanoacrylates. The primary role of these systems is to stop bleeding (hemostasis). In addition to high costs, fibrin adhesives feature a relatively weak adhesive strength and rapid breakdown, such that they can only be used for less severe injuries on tissue that is not stretched. Collagen and gelatin-based systems, such as FloSeal® work exclusively to attain hemostasis. Additionally, there is always a risk of infection with biological systems as fibrin and thrombin are extracted from human material and collagen and gelatin from animal material. Furthermore, biological materials must be stored in refrigeration, therefore they cannot be used for emergency care, such as in disaster areas, for military exercises, etc. In this case, trauma injuries can be treated with QuikClot® or QuikClot ACS+™, which are a mineral granulate that is applied to the wound in an emergency and causes coagulation by withdrawing water. QuikClot® produces a highly exothermic reaction, which leads to burns. QuikClot ACS+™ is gauze, into which salt is embedded. The system must be firmly pressed against the wound to stop bleeding.

WO 2009/106245 A2 highlights the production and use of polyurea systems as tissue adhesive. The systems revealed therein comprise at least two components. This involves an amino-functional aspartic acid ester and an isocyanate-functional prepolymer, which can be attained through the reaction of aliphatic polyisocyanates with polyester polyols. The two-component polyurea systems described can be used as tissue adhesive for closing wounds in human and animal cell structures. In doing so, a very positive adhesive result can be achieved.

To ensure that both components of the polyurea system can mix well, the viscosity of the components at 23° C. should—to the extent possible—be less than 10.000 mPa. Prepolymers with NCO functionalities have a respectively low viscosity of less than 3. If said prepolymers are used, it is necessary to use an aspartic acid ester with an amino functionality of more than two as a second component because otherwise a polymeric network cannot be produced. However, this is necessary so that said polyurea system or an adhesive joint consisting thereof has the desired mechanical properties, such as elasticity and strength. Moreover, there is a disadvantage to using difunctional aspartic acid ester, namely that the hardening time takes up to 24 hours, wherein the polyurea system itself remains tacky in many cases after this period, i.e. it is not “tack-free”. Furthermore, the resulting adhesives are primarily designed for topical applications and are not biologically degradable in the body within a short period, e.g. within 6 months or less. However, for applications within the body, an adhesive system should meet this requirement.

WO 2010/066356 highlights adhesive systems for medical applications, in which isocyanate-terminated prepolymers are reacted or hardened with secondary diamines. The disadvantages already mentioned in relation to WO 2009/106245 A2 occur in this case as well.

In addition to the actual bonding strength, the hardening time for tissue adhesives is therefore an essential parameter. If the adhesive hardens too quickly, the available time remaining for the user to apply it to the wound area to be bonded is possibly too little. In contrast, a prolonged hardening time is undesirable as this creates long waiting periods and the wound has to be immobilized during this time so that the wound area to be bonded does not separate again. An advisable hardening time may be specified, for example, from 1 to 5 minutes, wherein the optimal hardening time is ultimately aligned with the respective purpose of application. However, during this hardening time, the adhesive should remain workable for as long as possible.

It is obvious that adjusting the desired hardening time presents a challenge as the hardener has to be coordinated to the prepolymer or prepolymer composition to be hardened. In the process, the use of diamines for a certain prepolymer to be hardened can lead to rapid hardening; on the other hand, the aforementioned aspartate hardener may be too slow.

In this context, the goal of the invention was to provide a compound as a new hardener, wherein this compound should enable any hardening time for various polyurethane-urea systems. In doing so, the effect of this compound should be able to be adjusted to the hardening speed in certain areas to the extent possible. Furthermore, the guarantee of sufficient biological degradability following application in the animal or human body is desirable.

This task is solved through a compound of a formula (I)

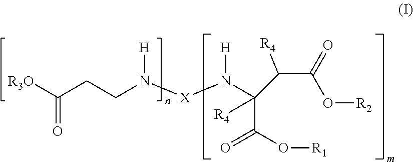

in which
R₁, R₂, R₃ are respectively independent equal or various organic radicals that have no Zerewitinoff active hydrogen,
R₄ are independently hydrogen, equal or different organic radicals, having no Zerewitinoff active hydrogen, or together form an unsaturated or aromatic ring, which can potentially contain heteroatoms, wherein R₄ have no Zerewitinoff active hydrogen,
X is a linear or branched, potentially even a substituted organic radical in the chain with heteroatom, which does not have Zerewitinoff active hydrogen,
n 0<n≦2 and
m 0≦m<2,
   wherein n+m=2.

In other words, the aforementioned compound has beta-amino acid ester groups as well as optionally aspartate ester groups with respectively variable shares. That means that n and m are not necessarily whole numbers, but rather the claimed composition can represent a mixture of various substituted compounds that fall under the aforementioned formula (I). In this regard, the mixture may naturally also contain a share of diaspartates, wherein this share is preferably less than 90 mol % in relation to the overall amount of substance of the compounds, particularly less than 75 mol %.

Surprisingly, it has been proven that compounds of this type demonstrate a high hardening speed when used as a hardener in a polyurethane-urea system. Thus, the hardening speed can be adapted to the desired degree in certain areas by means of variation of n or m. The tissue adhesives hardened in this way, for example on the basis of polyurethane urea, are tack free within a short period, which substantially simplifies their use.

In the configuration of the compound pursuant to the invention, the radicals R₁, R₂, R₃ are respectively independently linear or branched, particularly saturated, aliphatic C1 to C10 hydrocarbon radicals, preferably C2 to C18, particularly preferably C2 to C6, and very particularly preferably C2 to C4.

Furthermore, a radical X can be a linear, branched or cyclical organic C2 to C16 radical, preferably C3 to C14, particularly preferably C4 to C12. In this context, the radical X represents an aliphatic hydrocarbon radical in particular. Particularly preferable radicals are a 2-Methyl-pentamethylene radical, a Hexamethylene radical or an isophoryl radical, to name a few examples. Principally, mixtures of compounds can also be used with a different X.

To enable a most homogeneous hardening behavior, the radicals R₁, and R₂ can be respectively equal, wherein particularly the radicals R₁, R₂, and R₃ can be equal for a compound pursuant to the invention.

According to a preferred embodiment of the compound pursuant to the invention, 0<n<2 and 0<m<2, wherein n in particularly is 0.5 to 1.5, preferably 0.6 to 1.4, more preferably 0.7 to 1.3, particularly preferably 0.8 to 1.2, and very particularly preferably 0.9 to 1.1. In other words, this configuration of the invention relates to a mixture of compounds of said formula (I), for which statistically at least a portion of the compounds has a beta-amino acid group as well as an aspartate group. In the aforementioned number range of n, m approx. equal to 1, this mixture comprises nearly exclusively beta-amino acid ester modified aspartates from a statistical perspective. This is particularly beneficial as the reactivity of the hardener can be varied by adjusting n and m. Thus, the hardening speed, for example of a polyurethane-urea system, can be increased due to the fact that the share of beta-amino acid groups, i.e. the running figure n, is statistically increased in the case of the compound pursuant to the invention according to formula (I). In contrast, the share of aspartate groups, i.e. the running figure m, can be increased in the case of an excessive hardening speed.

The previously depicted adaption of the share of beta-amino acid groups and aspartate groups can be achieved by various means. Thus, by selecting an appropriate mixture ratio of reactants for producing the respective functional groups, the share of these groups can already be adjusted during production. This will be explained again further below. Further conceivable is mixing the pure di-beta-amino acid ester compound of formula (I) (i.e. n=2, m=0) or the pure di-aspartate compound of said formula (I) (i.e. n=0, m=2) with the pure beta-amino acid ester modified aspartate compound (i.e. n, m=1) of said formula (I) in an appropriate ratio. As explained above, this mixture can also comprise a share of di-aspartates and di-beta-amino acid esters, wherein this share is preferably less than 90 mol % in this case as well in relation to the overall amount of substance of the compounds, particularly less than 75 mol %.

A further object of the present invention relates to a process for producing a compound according to one of the claims 1 to 5, for which a diamine compound of a general formula (II)

H₂N—X—NH₂ (II)

is reacted with an acrylic acid ester of a general formula (III)

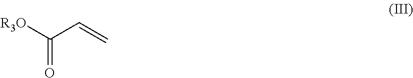

and, if desired, with a diester of an unsaturated dicarboxylic acid of a general formula (IV),

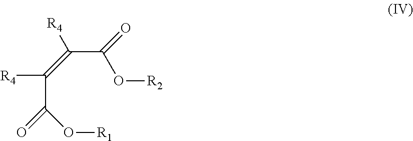

wherein n mol of acrylic acid ester and m mol of diester is used per mol of diamine compound,
and wherein
R₁, R₂, R₃ are respectively independent equal or various organic radicals that have no Zerewitinoff active hydrogen,
R₄ are independently hydrogen, equal or different organic radicals, having no Zerewitinoff active hydrogen, or form an unsaturated or aromatic ring, which can potentially contain heteroatoms, wherein R₄ have no Zerewitinoff active hydrogen,
X is a linear or branched, potentially even a substituted organic radical in the chain with heteroatoms, which does not have Zerewitinoff active hydrogen,
n 0<n≦2,
m0≦m<2,
and n+m=2.

Principally, all types of diamines can be used in the process pursuant to the invention. The do not demonstrate any Zerewitinoff active hydrogen atoms, aside from the two primary amino groups.

The Zerewitinoff active H atom indicates an acidic H atom or “active” H atom within the scope of the present invention. This can be determined in a conventional manner through reactivity with a respective Grignard reagent. The quantity of Zerewitinoff active H atoms is typically measured through the release of methane, which occurs according to a following reaction equation (formula 1) in a reaction of the substance to be tested with methylmagnesium bromide (CH₃—MgBr):

CH₃—MgBr+ROH→CH₄+Mg (OR)Br (1)

Zerewitinoff active H atoms typically originate from C—H acidic, organic groups, —OH, —SH, —NH₂ or —NHR with R as an organic radical, and —COOH.

As an acrylic acid ester, for example, those of the (Meth)acrylate type can be used. In this regard, for example, we can revert to C1 to C12 acrylates, particularly C1 to C10 acrylates, preferably C1 to C8 acrylates, more preferably C2 to C6 acrylates.

Maleic acid ester or the esters of a tetrahydrophthalic acid, for instance, can be viewed as diesters of an unsaturated dicarboxylic acid, particularly 3,4,5,6-Tetrahydrophthalic acid as well as combinations thereof. In this context, both radicals R₄ respectively correspond to a hydrogen atom in the case of maleic acid ester, wherein both radicals R₄ together form an unsaturated 6-ring in the case of tetrahydrophthalic acid.

Regardless of this, the diesters from C1 to C12 esters of the respective di-acid can be selected, particularly from the C1 to C8 esters, preferably from the 2 to C4 esters.

In the case of the process pursuant to the invention, n mol of acrylic acid ester and m mol of diester are used per mol of diamine compound. In this manner, the above described mixtures of the compound can be directly produced according to said formula (I). Thus, the adaption of the hardening speed described above can already be conducted by respectively controlling the production process.

The invention also relates to a compound of said formula (I), which can be produced according to the process pursuant to the invention.

A further object of the present invention relates to a polyurea system comprising the following components:
isocyanate functional prepolymers as a component A) can be achieved through a reaction of
aliphatic polyisocyanates A1) with
polyols A2), which may particularly have a number average molecular weight of ≧400 g/mol and an average OH functionality of 2 to 6,
a compound pursuant to the invention of said general formula (I) as component B),
potentially organic fillers, which may in particular have a viscosity measured according to DIN 53019 at 23° C. in the range of 10 to 6000 mPa, as a component C),
reaction products of isocyanate functional prepolymers according to component A) having compounds according to component B) and/or organic filler according to component C) potentially as component D), and
potentially water and/or a tertiary amine as a component E).

The polyurea systems pursuant to the invention are achieved by mixing prepolymers A) with the compound pursuant to the invention of said general formula (I) B) as well as potentially the components C), D), and/or E). In this regard, the ratio of free or blocked amino groups to free NCO groups is preferably 1:1.5, particularly preferably 1:1. Water and/or amine are added to component B) or C) in the process.

Isocyanate functional prepolymers A) can be achieved through a reaction of polyisocyanates A1) with polyols A2) potentially using catalysts and secondary and additional substances.

As a polyisocyanate A1), for example, monomeric aliphatic or cycloaliphatic di or triisocyanates, such as 1,4-butylene diisocyanate (BDI), 1,6-hexamethylene diisocyanate (HDI), Isophorone diisocyanate (IPDI), 2,2,4- and/or 2,4,4-trimethylhexa-methylene diisocyanate, the isomers bis-(4,4′-isocyanatocyclohexyl)-methane or their mixtures of any isomeric content, 1,4-cyclohexylene diisocyanate, 4-isocyanatomethyl-1,8-octane diisocyanate (nonane-triisocyanate), as well as alkyl-2,6-diisocyanatohexanoate (lysine diisocyanate) can be used with C1-C8 alkyl groups.

In addition to the aforementioned monomeric polyisocyanates A1), their higher molecular derived products may also be used in a uretdione, isocyanurate, urethane, allophanate, biuret, iminooxadiazindione or oxadiazine trione structure as well as its mixtures.

Polyisocyanates A1) of the aforementioned type are preferably used with exclusively aliphatically or cycloaliphatically bonded isocyanate groups or their mixtures.

It is likewise preferable if polyisocyanates A1) of the aforementioned type are used with an average NCO functionality of 1.5 to 2.5, preferably 1.6 to 2.4, more preferably 1.7 to 2.3, very particularly preferably 1.8 to 2.2, and particularly 2.

Hexamethylene diisocyanate is very particularly preferably used as a polyisocyanate A1).

One preferred embodiment of the polyurea system pursuant to the invention provides that the polyols A2) are polyester polyols and/or polyester-polyether polyols and/or polyether polyols. In this regard, polyester-polyether polyols and/or polyether polyols with an ethylene oxide share of between 60 to 90% by weight are particularly preferable.

It is also preferable if the polyols A2) have a number average molecular weight of 4000 to 8500 g/mol.

Suitable polyether ester polyols are preferably produced according to the state of the art through polycondensation from polycarboxylic acids, anhydrides of polycarboxylic acids, as well as esters of polycarboxylic acids with volatile alcohols, preferably C1 to C6 mono-ols, such as methanol, ethanol, propanol or butanol, with a molar-surplus, low-molecular and/or higher molecular polyol; wherein polyols containing ether groups are potentially used in mixtures with other polyols void of ether groups as a polyol.

Naturally, mixtures of higher molecular and low-molecular polyols may also be used for polyether-ester synthesis.

Such molar-surplus, low-molecular polyols are polyols with molar masses of 62 to 299 Da having 2 to 12 C atoms and hydroxyl functionalities of at least 2, which may also be branched or unbranched and their hydroxyl groups are primary or secondary. These low-molecular polyols may have ether groups as well. Typical substitutes are ethylene glycol, propanediol-1,2, propanediol-1,3, butanediol-1,4, butanediol-2, 3, 2-Methylpropanediol-1,3, pentanediol-1,5, hexanediol-1,6, 3-methyl pentanediol-1,5, 1, 8-octanediol, 1,10-decanediol, 1,12-dodecanediol, cyclohexanediol, diethylene glycol, triethylene glycol, and higher homologs, dipropylene glycol, Tripropylene glycol, and higher homologs, glycerin, 1,1,1-Trimethylolpropane, as well as oligo-tetrahydrofurans with hydroxyl end groups. Naturally, mixtures may also be used within these groups.

Molar-surplus higher molecular polyols are polyols with molar masses of 300 to 3000 Da, which can be obtained through ring-opening polymerization of epoxides, preferably ethylene and/or propylene oxide, as well as through acid-catalyzed, ring-opening polymerization of tetrahydrofuran. Either alkali hydroxide or double metal cyanide catalysts are used for ring-opening polymerization of epoxides.

All at least bi-functional molecules from the group of amines and the aforementioned low-molecular polyols can be used as starter for ring-opening epoxide polymerization. Typical substitutes are 1,1,1-trimethylolpropane, glycerin, o-TDA, ethylenediamine, propylene glycol-1,2, etc. as well as water, including their mixtures. Naturally, mixtures may also be used within this group of surplus higher molecular polyols.

The structuring of higher molecular polyols, if referring to hydroxyl group-terminated polyalkylene oxides from ethylene and/or propylene oxide, can occur statistically or in blocks, wherein mix blocks may also be contained.

Polycarboxylic acids are both aliphatic and aromatic carboxylic acids, which may be cyclical, linear, branched or unbranched and may have between 4 and 24 C atoms.

Examples are succinic acid, glutaric acid, adipic acid, azelaic acid, sebacic acid, 1,10-Decanedicarboxylic acid, 1,12-dodecandicarboxylic acid, phthalic acid, terephthalic acid, isophthalic acid, trimellitic acid, pyromellitic acid. Succinic acid, glutaric acid, adipic acid, sebacic acid, lactic acid, phthalic acid, terephthalic acid, isophthalic acid, trimellitic acid, and pyromellitic acid are preferable. Succinic acid, glutaric acid, and adipic acid are particularly preferable.

Furthermore, the group of polycarboxylic acids also comprises hydroxy carboxylic acids or their internal anhydrides, such as caprolactone, lactic acid, hydroxybutyric acid, ricinoleic acid, etc. This also includes monocarboxylic acids, particularly those having more than 10 C atoms, such as soy oil fatty acids, palm oil fatty acids, and peanut oil fatty acids, wherein their share of the overall reaction mixture forming the polyether-ester polyol does not exceed 10% by weight and, in addition, the resulting decreased functionality is compensated through the use of at least trifunctional polyols, whether on the part of low-molecular or high-molecular polyols.

Polyether-ester polyol is produced according to the state of the art at an elevated temperature in the range of 120 to 250° C. initially at normal pressure and subsequently by attaching a vacuum from 1 to 100 mbar, preferably, though not necessarily, through the use of an esterification or transesterification catalyst, wherein the reaction is completed to the extent that the acid value decreases to 0.05 to 10 mg KOH/g, preferably 0.1 to 3 mg KOH/g, and particularly preferably 0.15 to 2.5 mg KOH/g.

Furthermore, an inert gas can be used within the scope of a normal pressure stage prior to attaching a vacuum. Naturally, liquid or gaseous entrainers can be used alternatively or for individual stages of esterification. For example, the reaction water can be discharged using nitrogen as a carrier gas just as by using an azeotropic entrainer, such as benzole, toluene, xylol, dioxane, etc.

Naturally, mixtures of polyether polyols can be used with polyester polyols at any ratio.

Polyether polyols are preferably polyalkylene oxide polyethers based on ethylene oxide and potentially propylene oxide.

These polyether polyols are preferably based on di or higher functional starter molecules, such as two or higher functional alcohols or amines.

Examples of such starters are water (regarded as a diol), ethylene glycol, propylene glycol, butylene glycol, glycerin, TMP, sorbitol, pentaerythritol, triethanolamine, ammonia or ethylene diamine.

Polycarbonates having hydroxyl groups, preferably polycarbonate diols, can likewise be used with number average molecular weights of 400 to 8000 g/mol, preferably 600 to 3000 g/mol. These can be achieved through a reaction of carbonic acid derivatives, such as diphenyl carbonate, dimethyl carbonate or phosgene, with polyols, preferably diols.

Examples of these types of diols are ethylene glycol, 1,2- and 1,3-propanediol, 1,3- and 1,4-butanediol, 1,6-hexanediol, 1,8-octanediol, neopentyl glycol, 1,4-bishydroxymethyl cyclohexane, 2-methyl-1,3-propanediol, 2,2,4-trimethylpentanediol-1,3, dipropylene glycol, polypropylene glycols, dibutylene glycol, polybutylene glycols, bisphenol A and lactone-modified diols of the aforementioned type.

The polyisocynate A1) may be reacted with polyol A2) at an NCO/OH ratio of preferably 4:1 to 12:1, particularly preferably 8:1 for the production of prepolymer A), and subsequently the share of non-reacted polyisocyanate can be separated using suitable methods. Thin film distillation is usually used in this case, wherein prepolymers having residual monomer contents of less than 1% by weight, preferably less than 0.1% by weight, and particularly preferably less than 0.03% by weight can be achieved.

Stabilizers, such as benzoyl chloride, isophthaloyl chloride, dibutyl phosphate, 3-Chloropropionic acid or methyl tosylate, can potentially be used during production.

The reaction temperature when producing prepolymers A) is preferably 20 to 120° C., and more preferably 60 to 100° C.

The produced prepolymers have an average NCO content measured according to DIN EN ISO 11909 of 2 to 10% by weight, preferably 2.5 to 8% by weight.

According to a further embodiment of the polyurea system pursuant to the invention, prepolymers A) may have an average NCO functionality of 2 to 6, preferably 2.3 to 4.5, more preferably 2.5 to 4, very particularly preferably 2.7 to 3.5, and particularly 3.

Organic fillers of component C) may preferably be hydroxy functional compounds, particularly polyether polyols with repetitive ethylene oxide units.

It is beneficial if the fillers of component C) have an average OH functionality of 1.5 to 3, preferably 1.8 to 2.2, and particularly preferably 2.

For example, liquid polyethylene glycols, such as PEG 200 to PEG 600, their mono or dialkyl ethers, such as PEG 500 dimethyl ethers, liquid polyethers and polyester polyols, liquid polyesters, such as Ultramoll (Lanxess AG, Leverkusen, DE) as well as glycerin and its liquid derivatives, such as triacetine (Lanxess AG, Leverkusen, DE), can be used as organic fillers at 23° C.

The viscosity of the organic fillers—measured according to DIN 53019 at 23° C.—is preferably 50 to 4000 mPa, particularly preferably 50 to 2000 mPa.

In a preferred embodiment of the polyurea system pursuant to the invention, polyethylene glycols are used as organic fillers. They preferably have a number average molecular weight of 100 to 1000 g/mol, particularly preferably 200 to 400 g/mol.

To further reduce the average equivalent weight of the compound used overall for prepolymer grouping in relation to NCO reactive groups, it is possible to additionally produce reaction products of prepolymers A) with the compound B) pursuant to the invention of said general formula (I) and/or the organic fillers C)—if they are amino or hydroxy functional—in a separate preliminary reaction and to then use them as a higher molecular hardening component.

Ratios of isocyanate reactive groups to isocyanate groups of 50 to 1 to 1.5 to 1, particularly preferably 15 to 1 to 4 to 1 are preferably used in the preliminary extension.

The benefit of this modification through preliminary extension is that the equivalent weight and equivalent volume of the hardening component can be modified to greater extents. Thus, commercially available 2-chamber dispensing systems can be used for the application to achieve an adhesive system that can be added at existing ratios to the chamber volumes in the desired ratio of NCO reactive groups to NCO groups.

A further preferred embodiment of the polyurea system pursuant to the invention provides that the component E) contains a tertiary amine of a general formula (V),

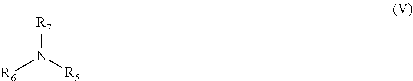

in which

R₅, R₆, R₇ may independently be alkyl or heteroalkyl radicals having heteroatoms in an alkyl chain or at their ends, or R₅ and R₆ can form an aliphatic, unsaturated or aromatic heterocycle together with the nitrogen atom bearing them, which can potentially contain additional heteroatoms.

These polyurea systems are distinguished by a particularly rapid hardening.

The compounds used in component E) can particularly preferably be tertiary amines selected from the group of triethanolamine, tetrakis (2-hydroxyethyl) ethylenediamine, N,N-dimethyl-2-(4-methylpiperazine-1-yl)ethanamine, 2-{[2-(dimethylamino)ethyl] (methyl) amino } ethanol, 3,3′, 3″-(1,3,5-triazinan-1,3,5-triyl)tris(N,N-dimethyl-propane-1-amine).

Very particularly high hardening speeds can also be achieved if component E) contains 0.2 to 2.0% by weight of water and/or 0.1 to 1.0% by weight of tertiary amine.

Naturally, pharmacologically active substances, such as analgesics with or without an anti-inflammatory effect, antiphlogistic, antimicrobially active substances, antimycotics, and antiparasitically active substances can be integrated in the polyurea systems as well.

The active substances may be pure active substances or in the form of a capsule to achieve, for example, a time-delayed release. Within the scope of the present invention, a number of types and classes of active substances can be used as medically active substances.

One such medically active substance may comprise, for example, a component releasing nitrogen monoxide under in vivo conditions, preferably L-arginine or a component containing or releasing L-arginine, particularly preferably L-arginine hydrochloride. Proline, ornithine and/or other biogenic intermediate stages, such as biogenic polyamines (spermine, spermidine, putrescine or bioactive artificial polyamines) may be used as well. As we know, these types of components promote the healing of wounds, wherein their continuous quantitatively nearly equal release is particularly tolerable for healing wounds.

Additional active substances usable pursuant to the invention comprise at least one substance selected from the group of vitamins or provitamins, carotinoides, analgesics, antiseptics, hemostyptics, antihistamines, antimicrobial metals or their salts, substances promoting the herbal healing of wounds or substance mixtures, herbal extracts, enzymes, growth factors, enzyme inhibitors as well as combinations thereof.

Particularly non-steroid analgesics, especially salicylic acid, acetylsalicylic acid and their derivatives, e.g. Aspirin®, aniline and its derivatives, acetaminophen e.g. Paracetamol®, anthranilic acid and its derivatives, e.g. mefenamine acid, pyrazole or its derivatives, methamizole, Novalgin®, phenazone, Antipyrin®, isopropylphenazone, and very particularly preferably aryl acetic acid, as well as its derivatives, heteroaryl acetic acids and its derivatives, arylpropionic acids and its derivatives, and heteroaryl propionic acids and its derivatives, e.g. Indometacin®, Diclophenac®, Ibuprofen®, Naxoprophen®, Indomethacin®, Ketoprofen®, Piroxicam® are suitable as analgesics.

As growth factors, the following should be mentioned in particular: aFGF (Acidic Fibroplast Growth Factor), EGF (Epidermal) Growth Factor), PDGF (Platelet Derived Growth Factor), rhPDGF-BB (Becaplermin), PDECGF (Platelet Derived Endothelial Cell Growth Factor), bFGF (Basic Fibroplast Growth Factor), TGF α; (Transforming Growth Factor alpha), TGF β (Transforming Growth Factor beta), KGF (Keratinocyte Growth Factor), IGF1/IGF2 (Insulin-Like Growth Factor), and TNF (Tumor Necrosis Factor).

Particularly those fat-soluble or water soluble vitamins, vitamin A, group of retinoids, provitamin A, group of carotenoids, particularly B-carotene, vitamin E, group of tocopherols, particularly α Tocopherol, β-Tocopherol, γ-Tocopherol, δ-Tocopherol, and α-Tocotrienol, β-Tocotrienol, γ-Tocotrienol, and δ-Tocotrienol, vitamin K, phylloquinone, particularly phytomenadione or herbal vitamin K, vitamin C, L-ascorbic acid, vitamin B 1, thiamin, vitamin B2, riboflavin, vitamin G, vitamin B3, niacin, nicotinic acid, and nicotinic acid amide, vitamin B5, pantothenic acid, provitamin B5, panthenol or dexpanthenol, vitamin B6, vitamin B7, vitamin H, biotin, vitamin B9, folic acid as well as combinations thereof are suitable as vitamins or provitamins.

As an antiseptic, it is necessary to use a medium that works as a germicide, bactericide, bacteriostatic, fungicide, virucide, virustatic, and/or general microbiocide.

Particularly those substances that are selected from the group of resorcinol, iodine, iodine povidone, chlorhexidine, benzalkonium chloride, benzoic acid, benzoyl peroxide or cethylpyridiniumchloride are suitable. Moreover, particularly antimicrobial metals can be used as antiseptics. Particularly silver, copper or zinc, as well as their salts, oxides or complexes can be used together or independently as antimicrobial metals.

In conjunction with the present invention, particularly chamomile extracts, hamamelis extracts, e.g. Hamamelis virginiana, calendula extract, aloe extract, e.g. aloe vera, Aloe barbadensis, Aloe ferox or Aloe vulgaris, green tea extracts, seaweed extract, e.g. red algae or green algae extract, avocado extract, myrrh extract, e.g. Commophora molmol, bamboo extracts as well as combinations thereof are referred to as herbal active substances promoting the healing of wounds.

The content of the active substances is primarily aligned with the medically necessary dose as well as tolerability with the remaining components of the composition pursuant to the invention.

The polyurea system pursuant to the invention is particularly suited to close, bond, adhere or cover cell tissue and particularly for stopping the discharge of blood or tissue fluids or closing leakages in cell tissue. It can be particularly preferably used for the application or production of a medium for closing, bonding, adhering or covering human or animal cell tissue. It can help to produce adhesive joints that are quick-hardening, strongly bonded to tissue, transparent, flexible, and bio-compatible.

Another object of the invention is a dispensing system with two chambers for a polyurea system pursuant to the invention, for which component A) is contained in one chamber, and components B) and potentially components C), D), and in another. E) of said polyurea system. Such a dispensing system is particularly suitable for applying the polyurea system as an adhesive to tissue.

### EXAMPLES

The present invention will be explained in further detail in the following using application examples.

### Methods:

### Molecular Weight:

The molecular weights were determined using gel permeation chromatography (GPC) as follows: The calibration was performed with polystyrene standards with molecular weights of Mp 1,000,000 to 162. Tetrahydrofuran p.A. was used as eluent. The following parameters were maintained during the double measurement: Degassing: Online-degasser; Flow rate: 1 ml/min.; Analysis period: 45 minutes; detectors: refractometer and UV detector; injection volume: 100 μl -200 μl. The calculation of the molar mass average values Mw; Mn and Mp as well as polydispersity Mw/Mn was performed using software. Baseline points and evaluation limits were defined according to DIN 55672 Part 1.

### NCO Content:

The NCO content was volumetrically determined according to DIN-EN ISO 11909 if not otherwise expressly stated.

### Viscosity:

The viscosity was determined according to ISO 3219 at 23° C.

### Residual Monomer Content:

The residual monomer content was determined according to DIN ISO 17025.

A Bruker DRX 700 device was used as an NMR.

### Synthesis of NCO-Terminated Prepolymers A:

465 g of HDI and 2.35 g of benzoyl chloride were presented in a 1 1 four-neck flask. 931.8 g of a trifunctional polyether (product of Bayer MaterialScience AG) with an ethylene oxide content of 71% and a propylene oxide content of 29%, respectively related to the overall alkylene oxide content, were added within 2 hours at 80° C. and subsequently stirred for 1 hour. The surplus HDI was then distilled off through thin film distillation at 130° C. and 0.13 mbar. We obtain 980 g (71%) of the prepolymer with an NCO content of 2.53% (equivalent weight: 1660 g/mol) and a viscosity of 4500 mPa/23° C. The residual monomer content was <0.03% HDI.

Synthesis of Polyol B with Lactide for Prepolymer C:

98.1 g of a poly(oxypropylene)triol started on glycerin with an OH value=400 mg KOH/g, 48.4 g of dilactide as well as 0.107 g of a DMC catalyst (produced according to EP-A 700 949) were presented in a 2 liter stainless steel pressure reactor under nitrogen and subsequently heated to 100° C. After 30 minutes of stripping with nitrogen at 0.1 bar, the temperature is increased to 130° C. and a mixture comprised of 701.8 g of ethylene oxide and 217.8 g of propylene oxide are then dispensed at this temperature within 130 minutes. After a subsequent reaction time of 45 minutes at 130° C., volatile shares are distilled off in a vacuum at 90° C. for 30 minutes and the reaction mixture is then cooled to room temperature.

### Product Properties:

OH value: 33.7 mg KOH/g

### Viscosity (25° C.): 1370 mPa

### Polydispersity (Mw/Mn): 1.13

### Synthesis of NCO-Terminated Prepolymer C:

293 g of HDI and 1.5 g of benzoyl chloride were presented in a 1 liter four-neck flask. 665.9 g of polyol B were added within 2 hours at 80° C. and subsequently stirred for 1 hour. The surplus HDI was then distilled off through thin film distillation at 130° C. and 0.13 mbar. The prepolymer is obtained with an NCO content of 2.37% (equivalent weight: 1772 g/mol). The residual monomer content was <0.03% HDI. Viscosity: 5740 mPa/23° C.

### Synthesis of the Hardener Pursuant to the Invention:

The hardeners pursuant to the invention were respectively synthesized based on a diamine compound. In the process, the following compounds were produced:

| | | | | Time until | | Time until | |
|---|---|---|---|---|---|---|---|
| | | | Equivalent | hardening with | | hardening with | |
| Hardener | Diamine/Acrylate | X | weight | prepolymer A | | prepolymer C | |
| HA1 | Dytek A/Ethyl acrylate | 0.5 | 200.72 g/mol | 4 | min. | 5 | min. |
| HA2 | Dytek A/Ethyl acrylate | 0.25 | 215.38 g/mol | 6 | min. | 8 | min. |
| HA3 | Dytek A/Ethyl acrylate | 0.125 | 226.72 g/mol | 6.5 | min. | 8.5 | min. |
| HB1 | Hexamethylenediamine/ | 0.5 | 208.95 g/mol | 3 | min. | 3 | min. |
| | Ethyl acrylate | | | | | | |
| HB2 | Hexamethylenediamine/ | 0.25 | 217.05 g/mol | 5 | min. | 5 | min. |
| | Ethyl acrylate | | | | | | |
| HB3 | Hexamethylenediamine/ | 0.125 | 221.34 g/mol | 5.5 | min. | 5.5 | min. |
| | Ethyl acrylate | | | | | | |
| HC1 | Isophorone diamine/ | 0.5 | 220.47 g/mol | 20 | min. | >5 | hours |
| | Ethyl acrylate | | | | | | |
| HC2 | Isophorone diamine/ | 0.25 | 239.32 g/mol | >5 | hours | >5 | hours |
| | Ethyl acrylate | | | | | | |
| HC3 | Isophorone diamine/ | 0.125 | 247.79 g/mol | >5 | hours | >5 | hours |
| | Ethyl acrylate | | | | | | |
| HD | Dytek A/Butyl acrylate | 0.375 | 216.21 g/mol | 6 | min. | 7 | min. |
| HE | Hexamethylenediamine/ | 0.375 | 215.38 g/mol | 5 | min. | 5 | min. |
| | Butyl acrylate | | | | | | |
| HF | Isophorone diamine/ | 0.375 | 239.32 g/mol | >5 | hours | >5 | hours |
| | Butyl acrylate | | | | | | |

For producing the aforementioned compounds, the following approach was taken respectively:

0.5 mol of the respective diamine was presented at room temperature (solid amines were presented as melt) and (1-x) mol of diethyl maleate (0≦x≦1) was added drop-wise over a period of 1 hour such that the temperature of the reaction mixture did not exceed 60° C. After 12 hours of stirring at room temperature, x mol of acrylate were added drop-wise over a period of 1 hour, such that the temperature of the reaction mixture did not exceed 60° C. After an abated exothermic reaction, the reaction mixture stirred for 24 hours at 60° C.

After cooling to room temperature, the reaction mixture was added to three parts of water and concentrated hydrochloric acid was added until a clear solution formed (pH value=1). The resulting solution was extracted three times with the same volume of ethyl acetate or dichloride methane and the stages were separated (organic stages are rejected). The aqueous stage was set through alkaline with concentrated caustic soda (pH value=10) and extracted with the same volume of ethyl acetate or dichloride methane once again three times and the stages were separated. The organic stage was dried using sodium sulfate and the solvent was removed in a vacuum. Yellow oils are obtained in quantitative yields.

### Hardening Tests:

1 eq of prepolymer A or C was presented in a plastic cup respectively with 1 eq of the hardener (HA1-3, HB1-3, HC1-3, HD, HE, HF) and mixed well for 30 seconds. The time was then measured until the mixture was tack free.

### In Vitro Attempt to Bond Tissue:

Respectively 1 eq of a hardener (HA1-3, HB1-3, HC1-3, HD, HE, HF) was added to 1 eq of prepolymer A and carefully stirred in a cup for 20 seconds. Directly thereafter, a thin layer of the polyurea system was applied to the muscle tissue to be bonded. The time during which the adhesive system still had a low viscosity was determined as the processing time, such that it could be applied to the tissue without difficulty.

The time, after which the polyurea system was no longer tacky (tack free time) was measured through bonding tests with a glass rod. In doing so, the glass rod was touched to the layer from the polyurea system. If it no longer remained bonded, the system was considered to be tack free. In addition, the bonding strength was determined, in which the ends of two pieces of muscle tissue (1=4 cm, h=0.3 cm, b=1 cm) were coated with the polyurea system 1 cm apart and adhered in an overlapping manner The bonding strength of the polyurea system was respectively tested through tension.

The results of the tissue bonding tests with prepolymer A are compiled in the following table:

| Hardener | Processing time | Tack free time | Adhesive strength |
|---|---|---|---|
| HA1 | 0:30 min. | 2:15 min. | + |
| HA2 | 3:00 min. | 3:10 min. | ++ |
| HA3 | 3:30 min. | 4:00 min. | ++ |
| HB1 | 0:33 min. | 1:50 min. | + |
| HB2 | 2:00 min. | 2:50 min. | ++ |
| HB3 | 3:45 min. | 3:15 min. | ++ |

The results prove that particularly the hardeners HA2, HA3, HB2, and HB3 combine a comparably long processing time with a short tack free time as well as good bonding strength. In contrast, the hardeners HA1 and HB1 are particularly rapidly tack free and can be processed for a respectively shorter period. Furthermore, these hardeners are distinguished by a minimally reduced bonding strength compared to the other hardeners.

1-15. (canceled)

16. A compound of a formula (I) 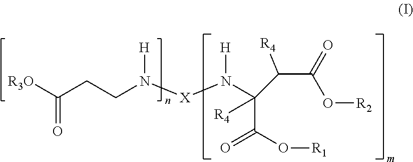
in which
R₁, R₂, R₃ are respectively independent equal or various organic radicals that have no Zerewitinoff active hydrogen,
R₄ are independently hydrogen, equal or different organic radicals, having no Zerewitinoff active hydrogen, or together form an unsaturated or aromatic ring, which can potentially contain heteroatoms, wherein R₄ have no Zerewitinoff active hydrogen,
X is a linear or branched organic radical, potentially substituted by a heteroatom in the chain, which does not have Zerewitinoff active hydrogen,
n 0<n≦2 and
m 0≦m<2,
wherein n+m=2.

17. The compound according to claim 16, wherein said radicals R₁, R₂, R₃ are respectively independently linear or branched, aliphatic C1 to C10 hydrocarbon radicals.

18. The compound according to claim 16, wherein the radical X is a linear, branched or cyclical, organic C2 to C 16 radical.

19. The compound according to claim 16, wherein said radicals R₁ and R₂ are equal.

20. The compound according to claim 16, wherein 0<n<2 and 0<m<2.

21. A process for producing the compound according to claim 16, comprising reacting a diamine compound of a general formula (II)
H₂N—X—NH₂ (II)
with an acrylic acid ester of a general formula (III) 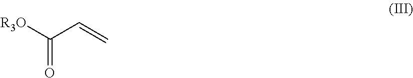
and, optionally, with a diester of an unsaturated dicarboxylic acid of a general formula (IV) 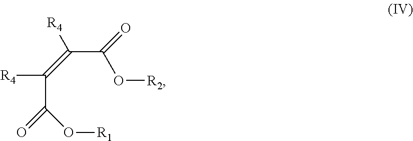
wherein n mol of acrylic acid ester and m mol of diester is used per mol of diamine compound, and wherein
R₁, R₂, R₃ are respectively independent equal or various organic radicals that have no Zerewitinoff active hydrogen,
R₄ are independently hydrogen, equal or different organic radicals, having no Zerewitinoff active hydrogen, or form an unsaturated or aromatic ring, which can potentially contain heteroatoms, wherein R₄ have no Zerewitinoff active hydrogen,
X is a linear or branched organic radical, potentially substituted by a heteroatom in the chain, which does not have Zerewitinoff active hydrogen,
n 0<n≦2,
m 0≦m<2
and n+m=2.

22. A polyurea system comprising
an isocyanate functional prepolymer as a component A) obtained by reaction of
an aliphatic polyisocyanate A1) with polyol A2),
a compound according to claim 16 as a component B),
obtionally organic fillers as a component C),
reaction products of isocyanate functional prepolymers according to component A) having compounds according to component B) and/or organic filler according to component C) potentially as component D), and
potentially water and/or a tertiary amine as a component E).

23. The polyurea system according to claim 22, wherein said polyol A2) comprise polyester polyols and/or polyester-polyether polyols and/or polyether polyols, particularly polyester-polyether polyols and/or polyether polyols having an ethylene oxide share of between 60 and 90% by weight.

24. The polyurea system according to claim 22, wherein said organic filler of a component C) are hydroxy functional compounds.

25. The polyurea system according to claim 22, wherein component E) comprises a tertiary amine of a general formula (V) 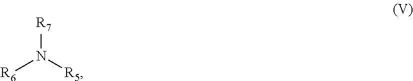
in which
R₅, R₆, R₇ may independently be alkyl or heteroalkyl radicals having heteroatoms in an alkyl chain or at their ends, or R₅ and R₆ can form an aliphatic, unsaturated or aromatic heterocycle together with the nitrogen atom bearing them, which can potentially contain additional heteroatoms.

26. The polyurea system according to claim 22, wherein said tertiary amine is selected from a group consisting of triethanolamine, tetrakis (2-hydroxyethyl) ethylenediamine, N,N-dimethyl-2-(4-methylpiperazine-1-yl)ethanamine, 2-{[2-(dimethylamino)ethyl](methyl)amino} ethanol, and 3,3′,3″-(1,3,5-Triazinan-1,3,5-triyl)tris(N,N-dimethyl-propane-1-amine).

27. The polyurea system according to claim 22, wherein said component E) contains 0.2 to 2.0% by weight of water and/or 0.1 to 1.0% by weight of tertiary amine.

28. A method comprising utilizing the polyurea system according to claim 22 for closing, bonding, adhering or covering cell tissue, or for closing leakages in cell tissue.

29. The method according to claim 28 wherein the cell tissue is human or animal cell tissue.

30. A dispensing system having two chambers for the polyurea/polyurethane system according to claim 22, wherein said component A) is contained in one chamber and said components B) and potentially said components C), D), and E) in another of said polyurea system.
